SEQUENCE LISTING (38 sequences)

SEQ ID NO: 1 - DNA, 996 bp, Escherichia coli
  CDS (1)..(996)
atgaagcaaacagtttatatcgccagccctgagagccagcaaattcacgtctggaatctgaatcatgaaggcgcactgacgctgacacaggttgtcgatgtgccggggcaggtgcagccgatggtggtcagcccggacaaacgttatctctatgttggtgttcgccctgagtttcgcgtcctggcgtatcgtatcgccccggacgatggcgcactgacctttgccgcagagtctgcgctgccgggtagtccgacgcatatttccaccgatcaccaggggcagtttgtctttgtaggttcttacaatgcgggtaacgtgagcgtaacgcgtctggaagatggcctgccagtgggcgtcgtcgatgtggtcgaggggctggacggttgccattccgccaatatctcaccggacaaccgtacgctgtgggttccggcattaaagcaggatcgcatttgcctgtttacggtcagcgatgatggtcatctcgtggcgcaggaccctgcggaagtgaccaccgttgaaggggccggcccgcgtcatatggtattccatccaaacgaacaatatgcgtattgcgtcaatgagttaaacagctcagtggatgtctgggaactgaaagatccgcacggtaatatcgaatgtgtccagacgctggatatgatgccggaaaacttctccgacacccgttgggcggctgatattcatatcaccccggatggtcgccatttatacgcctgcgaccgtaccgccagcctgattaccgttttcagcgtttcggaagatggcagcgtgttgagtaaagaaggcttccagccaacggaaacccagccgcgcggcttcaatgttgatcacagcggcaagtatctgattgccgccgggcaaaaatctcaccacatctcggtatacgaaattgttggcgagcaggggctactgcatgaaaaaggccgctatgcggtcgggcagggaccaatgtgggtggtggttaacgcacactaa

SEQ ID NO: 2 - protein, 331 aa, Escherichia coli
MKQTVYIASPESQQIHVWNLNHEGALTLTQVVDVPGQVQPMVVSPDKRYLYVGVRPEFRVLAYRIAPDDGALTFAAESALPGSPTHISTDHQGQFVFVGSYNAGNVSVTRLEDGLPVGVVDVVEGLDGCHSANISPDNRTLWVPALKQDRICLFTVSDDGHLVAQDPAEVTTVEGAGPRHMVFHPNEQYAYCVNELNSSVDVWELKDPHGNIECVQTLDMMPENFSDTRWAADIHITPDGRHLYACDRTASLITVFSVSEDGSVLSKEGFQPTETQPRGFNVDHSGKYLIAAGQKSHHISVYEIVGEQGLLHEKGRYAVGQGPMWVVVNAH

SEQ ID NO: 3 - DNA, 58 bp, Artificial Sequence
  Description of Artificial Sequence primer P1
catgaagcaaacagtttatatcgccagccctgagagcttacgccccgccctgccactc

SEQ ID NO: 4 - DNA, 59 bp, Artificial Sequence
  Description of Artificial Sequence primer P2
ttagtgtgcgttaaccaccacccacattggtccctggctgatgtccggcggtgcttttg

SEQ ID NO: 5 - DNA, 1096 bp, Artificial Sequence
  Description of Artificial Sequence 36 nucleotides from 5`-terminus of ybhE gene, Cm-resistance gene, 36 nucleotides from 3`-terminus of ybhE gene
catgaagcaaacagtttatatcgccagccctgagagcttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagccagggaccaatgtgggtggtggttaacgcacactaa

SEQ ID NO: 6 - DNA, 25 bp, Artificial Sequence
  Description of Artificial Sequence primer P3
tacaccgataccactatcggacaaa

SEQ ID NO: 7 - DNA, 19 bp, Artificial Sequence
  Description of Artificial Sequence primer P4
gaacgccagagacacgcgt

SEQ ID NO: 8 - DNA, 59 bp, Artificial Sequence
  Description of Artificial Sequence primer P5
ttaaatcaggtggctataaatgaactgggcaatgctgctgatgtccggcggtgcttttg

SEQ ID NO: 9 - DNA, 57 bp, Artificial Sequence
  Description of Artificial Sequence primer P6
atgaccacacgcgtgattgctctcgacttagacggcttacgccccgccctgccactc

SEQ ID NO: 10 - DNA, 59 bp, Artificial Sequence
  Description of Artificial Sequence primer P7
ttaacctatctcctgtaacgcgtgtctctggcgttcgctgatgtccggcggtgcttttg

SEQ ID NO: 11 - DNA, 57 bp, Artificial Sequence
  Description of Artificial Sequence primer P8
atgcagttaaaatttttaacggccagccacccaaaattacgccccgccctgccactc

SEQ ID NO: 12 - DNA, 1095 bp, Artificial Sequence
  Description of Artificial Sequence 36 nucleotides from 3`-terminus of ybhE gene, Cm-resistance gene, 36 nucleotides from 5`-terminus of ybhA gene
ttaaatcaggtggctataaatgaactgggcaatgctgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaagccgtctaagtcgagagcaatcacgcgtgtggtcat

SEQ ID NO: 13 - DNA, 1095 bp, Artificial Sequence
  Description of Artificial Sequence 36 nucleotides from 3`-terminus of ybhE gene, Cm-resistance gene, 36 nucleotides from 5`-terminus of ybhD gene
ttaacctatctcctgtaacgcgtgtctctggcgttcgctgatgtccggcggtgcttttgccgttacgcaccaccccgtcagtagctgaacaggagggacagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaattttgggtggctggccgttaaaaattttaactgcat

SEQ ID NO: 14 - DNA, 17 bp, Artificial Sequence
  Description of Artificial Sequence primer P9
cggccaggtggaagtgg

SEQ ID NO: 15 - DNA, 16 bp, Artificial Sequence
  Description of Artificial Sequence primer P10
ggctctcagggctggc

SEQ ID NO: 16 - DNA, 17 bp, Artificial Sequence
  Description of Artificial Sequence primer P11
cgagcaggggctactgc

SEQ ID NO: 17 - DNA, 16 bp, Artificial Sequence
  Description of Artificial Sequence primer P12
aacgcgcccctcgagg

SEQ ID NO: 18 - DNA, 68 bp, Artificial Sequence
  Description of Artificial Sequence primer P13
ttaaccgcgccacgctttatagcggttaatcagaccgaaagccacgttgtgtctcaaaatctctgatg

SEQ ID NO: 19 - DNA, 65 bp, Artificial Sequence
  Description of Artificial Sequence primer P14
aatgaaaaacatcaatccaacgcagaccgctgcctggcgctgaggtctgcctcgtgaagaaggtg

SEQ ID NO: 20 - DNA, 1286 bp, Artificial Sequence
  Description of Artificial Sequence 36 nucleotides from 3`-terminus of pgi gene, Km-resistance gene, 36 nucleotides from 5`-terminus of pgi gene
ttaaccgcgccacgctttatagcggttaatcagaccgaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccaggcagcggtctgcgttggattgatgtttttcatt

SEQ ID NO: 21 - DNA, 16 bp, Artificial Sequence
  Description of Artificial Sequence primer P15
ttaaccgcgccacgct

SEQ ID NO: 22 - DNA, 22 bp, Artificial Sequence
  Description of Artificial Sequence primer P16
aatgaaaaacatcaatccaacg

SEQ ID NO: 23 - DNA, 47 bp, Artificial Sequence
  Description of Artificial Sequence primer P17
gacaaagagctccacacaggaaacagctatgggagggcgtggtatgg

SEQ ID NO: 24 - DNA, 33 bp, Artificial Sequence
  Description of Artificial Sequence primer P18
ttagtagaattctcatgggcaccagtagatgtc

SEQ ID NO: 25 - DNA, 68 bp, Artificial Sequence
  Description of Artificial Sequence primer P19
ttacagcttagcgccttctacagcttcacgcgccaggaaagccacgttgtgtctcaaaatctctgatg

SEQ ID NO: 26 - DNA, 65 bp, Artificial Sequence
  Description of Artificial Sequence primer P20
atggcggtaacgcaaacagcccaggcctgtgacctggcgctgaggtctgcctcgtgaagaaggtg

SEQ ID NO: 27 - DNA, 1286 bp, Artificial Sequence
  Description of Artificial Sequence 36 nucleotides from 3`-terminus of eda gene, Km-resistance gene, 36 nucleotides from 5`-terminus of zwf gene
ttacagcttagcgccttctacagcttcacgcgccaggaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgccaggtcacaggcctgggctgtttgcgttaccgccat

SEQ ID NO: 28 - DNA, 23 bp, Artificial Sequence
  Description of Artificial Sequence primer P21
ttacagcttagcgccttctacag

SEQ ID NO: 29 - DNA, 18 bp, Artificial Sequence
  Description of Artificial Sequence primer P22
catggcggtaacgcaaac

SEQ ID NO: 30 - DNA, 35 bp, Artificial Sequence
  Description of Artificial Sequence primer P23
ctagtaagatctccctgtttgcaattaatcatcgg

SEQ ID NO: 31 - DNA, 35 bp, Artificial Sequence
  Description of Artificial Sequence primer P24
ctagtaagatctccctgttgacaattaatcatcgg

SEQ ID NO: 32 - DNA, 59 bp, Artificial Sequence
  Description of Artificial Sequence primer P25
tggcgatataaactgtttgcttcatgaatgctcctttcctgtgtgaaattgttatccgc

SEQ ID NO: 33 - DNA, 35 bp, Artificial Sequence
  Description of Artificial Sequence primer P26
ctagtaagatctgctgatgtccggcggtgcttttg

SEQ ID NO: 34 - DNA, 67 bp, Artificial Sequence
  Description of Artificial Sequence primer P27
gccaaaagcgactaattttagctgttacagtcagttgctaaatgcattacgccccgccctgccactc

SEQ ID NO: 35 - DNA, 1099 bp, Artificial Sequence
  Description of Artificial Sequence Cm-resistance gene, Ptac-3900 promoter
ttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcagatctccctgtttgcaattaatcatcggctcgtataatgtgtggaattgtgagcggataacaatttcacacagga

SEQ ID NO: 36 - DNA, 1099 bp, Artificial Sequence
  Description of Artificial Sequence Cm-resistance gene, Ptac-10000 promoter
ttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcagatctccctgttgacaattaatcatcggctcgtataatgtgtggaattgtgagcggataacaatttcacacagga

SEQ ID NO: 37 - DNA, 53 bp, Artificial Sequence
  Description of Artificial Sequence primer P28
gatatacatatgcaccaccaccaccaccacaagcaaacagtttatatcgccag

SEQ ID NO: 38 - DNA, 33 bp, Artificial Sequence
  Description of Artificial Sequence primer P29
agactaggatccttagtgtgcgttaaccaccac

(19) United States
(12) Patent Application Publication
(10) Pub. No.: US 2005/0191684 A1
(43) Pub. Date: Sep. 1, 2005

(54) Method for producing L-amino acids

(76) Inventors: Danila Vadimovich Zimenkov, Moscow (RU); Andrey Yurievich Gulevich, Moscow (RU); Aleksandra Yurievna Skorokhodova, Moscow (RU); Joanna Yosifovna Katashkina, Moscow (RU); Aleksandr Dmitrievich Kivero, Moscow (RU); Irina Vladimirovna Biryukova, Moscow (RU); Vera Georgievna Doroshenko, Moscow (RU); Sergei Vladimirovich Mashko, Moscow (RU)

Correspondence Address: CERMAK & KENEALY LLP;ACS LLC, 515 EAST BRADDOCK ROAD, SUITE B, ALEXANDRIA, VA, 22314, US

(21) Appl. No.: 11/063,223

(22) Filed: Feb. 23, 2005

Related U.S. Application Data
(60) Provisional application No. 60/604,698, filed on Aug. 27, 2004.

(30) Foreign Application Priority Data
Feb. 25, 2004 (RU) 2004105179
Jan. 26, 2005 (RU) 2005101700

(51) Int. Cl. C12Q 1/68; C12P 13/04; C12P 13/22; C07H 21/04; C12N 9/16; C12N 15/74

(52) U.S. Cl. 435/6; 435/106; 435/69.1; 435/196; 435/252.33; 435/488; 536/23.2

(57) ABSTRACT

A method for producing L-amino acids, such as L-tryptophan, L-phenylalanine, and L-tyrosine, using a bacterium of the Enterobacteriaceae family is provided. The L-amino acid productivity of said bacterium is increased by enhancing an activity of 6-phosphogluconolactonase, which is encoded by the pgl gene (ybhE ORF).

This application claims the benefit of application Ser. No. 60/604,698, filed Aug. 27, 2004, under 35 U.S.C. §119(e).

### BACKGROUND OF THE INVENTION

1. Field of the Invention

The present invention relates to a method for producing L-amino acids by fermentation using a microorganism. Specifically, the present invention relates to a method for producing aromatic amino acids such as L-tryptophan, L-phenylalanine, and L-tyrosine.

2. Brief Description of the Related Art

The pentose phosphate pathway (PPP) is an important part of the central metabolism of a majority of organisms. In the oxidative branch of PPP, the synthesis of NADPH takes place, and phosphorylated carbohydrates of the non-oxidative branch of PPP are precursors for nucleotide biosynthesis (ribose-5-phosphate), aromatic amino acids and vitamins (erythrose-5-phosphate). Erythrose 4-phosphate (E4p) are the essential precursors of the common biosynthetic pathway for aromatic L-amino acids. Optimization of the specific pathways of phosphoenolpyruvate (PEP) and E4p biosynthesis can, therefore, improve production of aromatic L-amino acids.

The oxidative branch of PPP includes three reactions. The first and third reactions are catalyzed by the well-known enzymes glucose-6-phosphate dehydrogenase (EC 1.1.1.49) and gluconate-6-phosphate dehydrogenase (EC 1.1.1.44), which are encoded by the zwf and gnd genes, respectively. The second reaction is the hydrolysis of 6-phosphogluconolactone to 6-phosphogluconate (Escherichia coli and Salmonella, Second Edition, Editor in Chief: F. C. Neidhardt, ASM Press, Washington D.C., 1996). An enzyme which catalyzes this reaction has been detected in several organisms including, for example, human (Collard, F., et al, FEBS Lett., 459:2, 223-6 (1999)), Trypanosoma brucei (Duffieux, F., et al, J. Biol. Chem., 275:36, 27559-65 (2000)), Plasmodium berghei (Clarke, J. L., et al, Eur. J. Biochem., 268:7, 2013-9 (2001)), Pseudomonas aeroginosa (Hager P. W. et al, J. Bacteriol., 182:14,3934-41 (2000)), Pseudomonas putida (Petruschka, L., et al, FEMS Microbiol. Lett., 215:1, 89-95 (2002)), however it is also known that the reaction can go spontaneously.

δ-6-Phosphogluconolactone, one of the products of the reaction catalyzed by glucose-6-phosphate dehydrogenase, is able to isomerize to γ-6-phosphogluconolactone in the course of intermolecular rearrangement. Only δ-6-phosphogluconolactone is able to hydrolyze to 6-phosphogluconate spontaneously, and exactly that reaction is catalyzed by known 6-phosphogluconolactonases (EC 3.1.1.31) (Miclet E. et al., J Biol. Chem., 276:37, 34840-46 (2001)). The pgl gene from E. coli, presumably encoding 6-phosphogluconolactonase, was mapped on the chromosome of E. coli between att-λ and chlD gene (in the modern databases—modC gene). Mutants of E. coli (pgl⁻) exhibit “maltose-blue” phenotype (Kupor, S. R. and Fraenkel, D. G., J. Bacteriol., 100:3, 1296-1301 (1969)) which is a distinctive feature of strains which accumulate maltodextrine (Adhya S. and Schwartz M., J Bacteriol, 108:2, 621-626 (1971)).

But at present time, neither the sequence nor the exact location of the pgl gene on the chromosome of E. coli is known. Enzymes having the activity of 6-phosphogluconolactonase from E. coli have not been isolated and there are no reports linking enhancement of 6-phosphogluconolactonase activity in the cell of a L-amino acid-producing bacterium with an increase in L-amino acid production.

### SUMMARY OF THE INVENTION

An object of the present invention is to provide 6-phosphogluconolactonase from E. coli, to enhance the productivity of L-amino acid-producing strains, and to provide a method for producing L-amino acids using the strain.

This object was achieved by identifying the fact that ybhE open reading frame (ORF) of E. coli strain K-12 encodes 6-phosphogluconolactonase and enhanced expression of the ybhE ORF (pgl gene) can enhance L-amino acid production by the respective L-amino acid producing strains. Thus, the present invention has been completed.

It is an object of the present invention to provide an L-amino acid producing bacterium, wherein the bacterium has been modified to enhance an activity of 6-phosphogluconolactonase It is a further object of the present invention to provide the bacterium as described above, wherein the bacterium belongs to the Enterobacteriaceae family, and wherein said bacterium is selected from the group consisting of Escherichia, Erwinia, Providencia, and Serratia.

It is a further object of the present invention to provide the bacterium as described above, wherein the activity of 6-phosphogluconolactonase is enhanced by modifying an expression control sequence of the 6-phosphogluconolactonase gene on the chromosome of the bacterium so that the expression of the gene is enhanced.

It is a further object of the present invention to provide the bacterium as described above, wherein a native promoter of said gene is replaced with a more potent promoter.

It is a further object of the present invention to provide the bacterium as described above, wherein the 6-phosphogluconolactonase gene is derived from a bacterium belonging to the genus Escherichia.

It is a further object of the present invention to provide the bacterium as described above, wherein the 6-phosphogluconolactonase gene is selected from the group consisting of:
(a) a DNA comprising a nucleotide sequence of the nucleotides 1 to 993 in SEQ ID NO: 1; and
(b) a DNA which is hybridizable with a nucleotide sequence of the nucleotides 1 to 993 in SEQ ID NO: 1 or a probe which can be prepared from the nucleotide sequence under stringent conditions and encodes a protein having an activity of 6-phosphogluconolactonase.

It is a further object of the present invention to provide the bacterium as described above, wherein the stringent conditions comprise washing for 15 minutes at 60° C., at a salt concentration corresponding to 1×SSC and 0.1% SDS.

It is a further object of the present invention to provide the bacterium described above, wherein the bacterium is further modified to have enhanced expression of the ybhE open reading frame.

It is a further object of the present invention to provide the bacterium described above, wherein L-amino acid is an aromatic L-amino acid selected from the group consisting of L-tryptophan, L-phenylalanine, and L-tyrosine.

It is a further object of the present invention to provide a method for producing aromatic L-amino acids comprising cultivating the bacterium as described above in a culture medium and collecting from the culture medium said L-amino acid.

It is a further object of the present invention to provide the method described above, wherein the L-amino acid is an aromatic amino acid selected from the group consisting of L-tryptophan, L-phenylalanine, and L-tyrosine.

It is a further object of the present invention to provide the method described above, wherein the bacterium has enhanced expression of genes for aromatic amino acid biosynthesis.

The method for producing an L-amino acid includes production of L-tryptophan using L-tryptophan-producing bacterium, wherein the activity of the protein of the present invention is enhanced. The method for producing an L-amino acid also includes production of L-phenylalanine using L-phenylalanine-producing bacterium, wherein the activity of the protein of the present invention is enhanced. The method for producing L-amino acid further includes production of L-tyrosine using L-tyrosine-producing bacterium, wherein the activity of the protein of the present invention is enhanced.

### BRIEF DESCRIPTION OF DRAWINGS

FIG. 1 shows the structure of bacterial native DNA region around ybhE ORF.

FIG. 2 shows the structure of bacterial DNA region with deleted ybhE ORF.

FIG. 3 shows the structure of bacterial DNA region with deleted ybhA ORF.

FIG. 4 shows the structure of bacterial DNA region with deleted ybhD ORF.

FIG. 5 shows the structure of bacterial DNA region with deleted pgi gene.

FIG. 6 shows the structure of bacterial DNA region with deleted zwf-edd-eda operon.

FIG. 7 shows the structure of bacterial DNA region with artificial promoter region (P_{tac*}) upstream of pgl gene (ybhE ORF).

FIG. 8 shows the gel separation of (His)6-YbhE protein and purification. A. Crude extracts of BL21 (DE3)[pET-HTybhE] strain. Lines 1, 2, 9—Protein Molecular Weight Marker; lines 3,4—total cell protein of the strain without and with IPTG induction; lines 5,6—soluble fraction of the strain without and with IPTG induction; lines 7, 8—insoluble fraction of the strain without and with IPTG induction. B. Line 1—total cell proteins of BL21(DE3)[pET-HTybhE]; lines 2, 3, 4, 6—increasing concentration of the purified (His)6-YbhE; line 5—Protein Molecular Weight Marker.

### DESCRIPTION OF THE PREFERRED EMBODIMENTS

According to the present invention, an L-amino acid producing bacterium is described, wherein the bacterium has been modified to enhance an activity of 6-phosphogluconolactonase. The term “activity of 6-phosphogluconolactonase” means an activity to catalyze the hydrolysis reaction of 6-phosphogluconolacton to 6-phosphogluconate. Activity of 6-phosphogluconolactonase is measured by the method described by, for example, Kupor, S. R. and Fraenkel, D. G. (J. Bacteriol., 100:3, 1296-1301 (1969)). The gene encoding 6-Phosphogluconolactonase may be the ybhE gene of Escherichia coli or a homologue thereof.

As the gene encoding 6-phosphogluconolactonase of Escherichia coli (EC number 3.1.1.31), pgl gene including ybhE ORF is stated (nucleotide numbers 797809 to 798804 in the sequence of GenBank accession NC_{—}000913.1, gi:16128735). The ybhE ORF is located between ybhA and ybhD ORFs on the chromosome of E. coli strain K12. Therefore, pgl gene can be obtained by PCR (polymerase chain reaction; refer to White, T. J. et al., Trends Genet., 5, 185 (1989)) utilizing primers prepared based on the nucleotide sequence of the gene.

The pgl gene from Escherichia coli is exemplified by a DNA which comprises the following DNA (a) or (b):
(a) a DNA which comprises a nucleotide sequence of the nucleotides 1 to 993 in SEQ ID NO: 1; or
(b) a DNA which is hybridizable with a nucleotide sequence of the nucleotides 1 to 993 in SEQ ID NO:1, or a probe which can be prepared from the nucleotide sequence, under stringent conditions and codes for a protein having an activity of 6-phosphogluconolactonase.

The DNA encoding proteins of the present invention includes a DNA encoding the protein which includes deletion, substitution, insertion or addition of one or several amino acids in one or more positions on the protein (A) as long as they do not lose the activity of the protein. Although the number of “several” amino acids differs depending on the position in the three-dimensional structure of the protein or the type of amino acid residues, it may be 2 to 30, preferably 2 to 20, and more preferably 2 to 10 for the protein (A).

The protein of the present invention, having the above-described deletions, substitutions, insertions, or additions of one or several amino acids, is at least 70% homologous to the protein of SEQ ID NO:2. Percent homology of the protein is measured by comparing the variant sequence to that in SEQ ID NO:2 over the length of the entire sequence and determining the number of like residues. The protein of the present invention is at least 70% homologous to the protein of SEQ ID NO:2, more preferably at least 80% homologous, even more preferably at least 90% homologous, and most preferably at least 95% homologous to the protein of SEQ ID NO:2. Percent homology of a protein or DNA can also be evaluated by known calculation methods such as BLAST search, FASTA search and CrustalW. BLAST (Basic Local Alignment Search Tool) is the heuristic search algorithm employed by the programs blastp, blastn, blastx, megablast, tblastn, and tblastx; these programs ascribe significance to their findings using the statistical methods of Karlin, Samuel and Stephen F. Altschul (“Methods for assessing the statistical significance of molecular sequence features by using general scoring schemes”. Proc. Natl. Acad. Sci. USA, 1990, 87:2264-68; “Applications and statistics for multiple high-scoring segments in molecular sequences”. Proc. Natl. Acad. Sci. USA, 1993, 90:5873-7). FASTA search method described by W. R. Pearson (“Rapid and Sensitive Sequence Comparison with FASTP and FASTA”, Methods in Enzymology, 1990 183:63-98). ClustalW method described by Thompson J. D., Higgins D. G. and Gibson T. J. (“CLUSTAL W: improving the sensitivity of progressive multiple sequence alignment through sequence weighting, position-specific gap penalties and weight matrix choice”, Nucleic Acids Res. 1994, 22:4673-4680).

Changes to the protein defined in (A) such as those described above are typically conservative changes so as to maintain the activity of the protein. Substitution changes include those in which at least one residue in the amino acid sequence has been removed and a different residue inserted in its place. Examples of amino acids which may be substituted for an original amino acid in the above protein and which are regarded as conservative substitutions include: Ala substituted with ser or thr; arg substituted with gin, his, or lys; asn substituted with glu, gin, lys, his, asp; asp substituted with asn, glu, or gin; cys substituted with ser or ala; gin substituted with asn, glu, lys, his, asp, or arg; glu substituted with asn, gin, lys, or asp; gly substituted with pro; his substituted with asn, lys, gin, arg, tyr; ile substituted with leu, met, val, phe; leu substituted with ile, met, val, phe; lys substituted with asn, glu, gin, his, arg; met substituted with ile, leu, val, phe; phe substituted with trp, tyr, met, ile, or leu; ser substituted with thr, ala; thr substituted with ser or ala; trp substituted with phe, tyr; tyr substituted with his, phe, or trp; and val substituted with met, ile, leu.

The DNA encoding substantially the same protein as the protein defined in (A) may be obtained by, for example, modification of nucleotide sequence encoding the protein defined in (A) using site-directed mutagenesis so that one or more amino acid residue will be deleted, substituted, inserted, or added. Such modified DNA can be obtained by conventional methods using treatment with reagents and conditions generating mutations. Such treatment includes treatment the DNA encoding proteins of present invention with hydroxylamine or treatment the bacterium harboring the DNA with UV irradiation or reagent such as N-methyl-N′-nitro-N-nitrosoguanidine or nitrous acid.

The DNA encoding proteins of the present invention includes variants which can be found in the different strains and variants of bacteria belonging to the genus Escherichia according to natural diversity. The DNA encoding such variants can be obtained by isolating the DNA, which hybridizes with pgl gene or part of the gene under the stringent conditions, and which encodes the protein having activity of 6-phosphogluconolactonase. The term “stringent conditions” referred to herein is a condition under which a so-called specific hybrid is formed, and a non-specific hybrid is not formed. For example, stringent conditions include conditions under which DNAs having high homology, for instance DNAs having homology no less than 70%, preferably no less than 80%, more preferably no less than 90%, most preferably no less than 95% to each other, are hybridized. Alternatively, stringent conditions are exemplified by conditions which comprise ordinary conditions of washing in Southern hybridization, e.g., 60° C., approximately 1×SSC, 0.1% SDS, preferably 0.1×SSC, 0.1% SDS. Duration of washing depends on the type of membrane used for blotting and, as a rule, is recommended by the manufacturer. For example, recommended duration of washing of the Hybond™ N+ nylon membrane (Amersham) under stringent conditions is 15 minutes. Preferably, washing may be performed 2 to 3 times.

As a probe for the DNA that codes for variants and hybridizes with pgl gene, a partial sequence of the nucleotide sequence of SEQ ID NO: 1 can also be used. Such a probe may be prepared by PCR using oligonucleotides based on the nucleotide sequence of SEQ ID NO: 1 as primers, and a DNA fragment containing the nucleotide sequence of SEQ ID NO: 1 as a template. When a DNA fragment in a length of about 300 bp is used as the probe, the conditions of washing for the hybridization consist of, for example, 50° C., 2×SSC, and 0.1% SDS.

Transformation of a bacterium with a DNA encoding a protein means introduction of the DNA into bacterium cell for example by conventional methods to increase expression of the gene encoding the protein of the present invention and to enhance the activity of the protein in the bacterial cell.

A bacterium of the present invention is L-amino acid-producing bacterium belonging to the Enterobacteriaceae family having enhanced activities of a protein, which enhances the productivity of the target L-amino acid. Preferably, the bacterium of the present invention is an aromatic L-amino acid-producing bacterium, specifically belonging to the genus Escherichia that has enhanced activity of the protein of the present invention. More preferably, the bacterium of the present invention is an aromatic L-amino acid-producing bacterium, such as L-tryptophan-producing bacterium, specifically belonging to the genus Escherichia, wherein the bacterium has been modified to enhance an activity of 6-phosphogluconolactonase. More preferably, the bacterium of the present invention harbors the DNA comprising the pgl gene (ybhE ORF) with a modified expression control sequence on the chromosome of the bacterium and has enhanced ability to produce L-tryptophan.

“L-amino acid-producing bacterium” means a bacterium, which has an ability to cause accumulation of the L-amino acid in a medium when the bacterium of the present invention is cultured in the medium. The L-amino acid-producing ability may be imparted or enhanced by breeding. The term “L-amino acid-producing bacterium” used herein also means a bacterium, which is able to produce and cause accumulation of a L-amino acid in a culture medium in amount larger than a wild-type or parental strain, and preferably means that the microorganism is able to produce and cause accumulation in a medium of an amount not less than 0.5 g/L, more preferably not less than 1.0 g/L of target L-amino acid. L-amino acids include L-alanine, L-arginine, L-asparagine, L-aspartic acid, L-cysteine, L-glutamic acid, L-glutamine, L-glycine, L-histidine, L-isoleucine, L-leucine, L-lysine, L-methionine, L-phenylalanine, L-proline, L-serine, L-threonine, L-tryptophan, L-tyrosine, and L-valine, and preferably includes aromatic L-amino acids, such as L-tryptophan, L-phenylalanine, and L-tyrosine.

The Enterobacteriaceae family includes bacteria belonging to the genera Escherichia, Enterobacter, Erwinia, Klebsiella, Pantoea, Providencia, Salmonella, Serratia, Shigella, Morganella. Enterobacter, Erwinia, Escherichia, Klebsiella, Providencia, Salmonella, Serratia, Shigella etc. Specifically, those classified into the Enterobacteriaceae according to the taxonomy used in the NCBI (National Center for Biotechnology Information) database (http://www.ncbi.nlm.nih.gov/htbinpost/Taxonomy/wgetorg?mode=Tree&id=1236&lvl=3&keep=1&srchmode=1&unlock) can be used. The genus Escherichia is preferred.

The phrase “a bacterium belonging to the genus Escherichia” means that the bacterium is classified in the genus Escherichia according to the classification known to a person skilled in the art of microbiology. Examples of a microorganism belonging to the genus Escherichia as used in the present invention include, but are not limited to, Escherichia coli (E. coli).

The bacterium belonging to the genus Escherichia that can be used in the present invention is not particularly limited, however for example, bacteria described by Neidhardt, F. C. et al. (Escherichia coli and Salmonella typhimurium, American Society for Microbiology, Washington D.C., 1208, Table 1) are encompassed by the present invention. Examples of wild-type strains of Escherichia coli include, but are not limited to, the K12 strain and derivatives thereof, Escherichia coli MG1655 strain (ATCC No. 47076), and W3110 strain (ATCC No. 27325). These strains are available from the American Type Culture Collection (ATCC, Address: 12301 Parklawn Drive, Rockville Md. 20852, United States of America).

The term “a bacterium belonging to the genus Pantoea” means that the bacterium is classified as the genus Pantoea according to the classification known to a person skilled in the art of microbiology. Some species of Enterobacter agglomerans have been recently re-classified into Pantoea agglomerans, Pantoea ananatis, Pantoea stewartii or the like, based on nucleotide sequence analysis of 16S rRNA etc.

The term “modified to enhance an activity of 6-phosphogluconolactonase” means that the activity per cell has become higher than that of a non-modified strain, for example, a wild-type strain. The activity of 6-phosphogluconolactonase can be measured by using Collard's method (FEBS Letters 459 (1999) 223-226). An example is when the number of 6-phosphogluconolactonase molecules per cell increases, the specific activity per 6-phosphogluconolactonase molecule increases and so forth. Furthermore, as a wild-type strain that serves as an object for comparison, for example, the Escherichia coli K-12 is encompassed. As a result of enhanced intracellular activity of 6-phosphogluconolactonase, the amount of L-amino acid such as L-tryptophan, which is accumulates in a medium, is increased.

Enhancement of 6-phosphogluconolactonase activity in a bacterial cell is attained by increasing expression of a gene encoding 6-phosphogluconolactonase. (pgl gene) As the 6-phosphogluconolactonase genes, a gene derived from a bacterium from Enterobacteriaceae is encompassed. Expression of the pgl gene can be enhanced by, for example, increasing the copy number of the pgl gene in cells using genetic recombination techniques. For example, a recombinant DNA can be prepared by ligating a gene fragment containing the pgl gene to a vector, preferably a multi-copy vector, which is operable in cells of a host microorganism, and introducing the resulting vector into the cells of the host microorganism.

When the pgl gene of Escherichia coli is used, the pgl gene (ybhE) may be obtained by, for example, the PCR method (polymerase chain reaction, refer to White, T. J. et al., Trends Genet., 5, 185 (1989)) using primers designed based on a nucleotide sequence of SEQ ID NO: 1, using chromosomal DNA of Escherichia coli as a template. The pgl gene from other microorganisms may also be used, and can be obtained from their chromosomal DNA or chromosomal DNA library by PCR using oligonucleotide primers designed based on a sequence of their pgl gene or a homologous sequence thereof of pgl gene or the 6-phosphogluconolactonase protein from a different species of microorganisms, or by hybridization using an oligonucleotide probe prepared based on such sequence information. A chromosomal DNA can be prepared from a microorganism serving as a DNA donor by, for example, the method of Saito and Miura (refer to H. Saito and K. Miura, Biochem. Biophys. Acta, 72, 619 (1963), Text for Bioengineering Experiments, Edited by the Society for Bioscience and Bioengineering, Japan, pp. 97-98, Baifukan, 1992).

Then, the pgl gene is ligated to a vector DNA operable in cells of the host microorganism to prepare a recombinant DNA. Preferably, vectors autonomously replicable in cells of the host microorganism are used.

Examples of vectors autonomously replicable in Escherichia coli include pUC19, pUC18, pHSG299, pHSG399, pHSG398, pACYC184, (PHSG and pACYC are available from Takara Bio), RSF1010, pBR322, pMW219 (pMW is available from Nippon Gene), and so forth.

In order to prepare a recombinant DNA by ligating the pgl gene and any of the vectors mentioned above, the vector and a fragment containing the pgl gene are digested with restriction enzymes and ligated with each other, usually by using a ligase such as a T4 DNA ligase.

To introduce a recombinant DNA prepared as described above into a microorganism, any known transformation methods reported so far can be employed. For example, a method of treating recipient cells with calcium chloride so as to increase the permeability of DNA, which has been reported for Escherichia coli (Mandel, M. and Higa, A., J. Mol. Biol., 53, 159 (1970)), and a method of using competent cells prepared from growing cells to introduce a DNA, which has been reported for Bacillus subtilis (Duncan, C. H., Wilson, G. A. and Young, F. E., Gene, 1, 153 (1977)) can be employed. In addition to these methods, a method of introducing a recombinant DNA into protoplast- or spheroplast-like recipient cells, which haved been reported to be applicable to Bacillus subtilis, actinomycetes, and yeasts (Chang, S. and Choen, S. N., Molec. Gen. Genet., 168, 111 (1979); Bibb, M. J., Ward, J. M. and Hopwood, O. A., Nature, 274, 398 (1978); Hinnen, A., Hicks, J. B. and Fink, G. R., Proc. Natl. Sci., USA, 75, 1929 (1978)), can be employed.

The copy number of the pgl gene can also be increased by integrating multiple copies of pgl the gene on a chromosomal DNA of a microorganism. In order to integrate multiple copies of the pgl gene on a chromosomal DNA of a microorganism, homologous recombination can be performed by targeting a sequence on a chromosomal DNA in multiple copies. As a sequence which exists on a chromosomal DNA in multiple copies, repetitive DNA and inverted repeats existing at an end of a transposon can be used as a sequence in which multiple copies exist on a chromosomal DNA. Alternatively, as disclosed in JP2-109985A, it is also possible to incorporate the pgl gene into a transposon, and allow it to be transferred so that multiple copies of the gene are integrated into the chromosomal DNA. Integration of the pgl gene into the chromosome can be confirmed by southern hybridization using a probe having a partial sequence of the pgl gene.

The bacterium of the present invention includes one wherein the activity of the protein of the present invention is enhanced by alteration of a expression control sequence of DNA encoding a protein as defined in (A) or (B) on the chromosome of the bacterium (WO00/18935). The enhancement of gene expression can be achieved by placing the DNA of the present invention under the control of a more potent promoter instead of the native promoter. For example, the lac promoter, trp promoter, trc promoter, tac promoter, PR promoter and so forth are known as strong promoters. The term “native promoter” means a DNA region present in the wild-type organism, located upstream of the open reading frame (ORF) of the gene and having a function of promoting transcription of the gene. Strength of a promoter is defined by the frequency of acts of RNA synthesis initiation. Methods for evaluating the strength of the promoter are described in, for example, Deuschle U., Kammerer W., Gentz R., Bujard H. (Promoters in Escherichia coli: a hierarchy of in vivo strength indicates alternate structures. EMBO J. 1986, 5, 2987-2994). A method for evaluating potency of promoter and examples of potent promoters are disclosed in Goldstein et al. (Prokaryotic promoters in biotechnology. Biotechnol. Annu. Rev., 1995, 1, 105-128).

Enhancing the translation can be achieved by introducing into the DNA of the present invention a more efficient Ribosome Binding Site (RBS) in place of the native RBS sequence. The RBS sequence is a region upstream of the start codon of the mRNA which interacts with the 16S RNA of ribosome (Shine J. and Dalgarno L., Proc. Natl. Acad. Sci. USA, 1974, 71, 4, 1342-6). The term “native RBS sequence” means RBS sequence presented in the wild-type organism. The RBS sequence of the gene 10 from phage T7 can be exemplified as an efficient RBS sequence (Olins P. O. et al, Gene, 1988, 73, 227-235).

The bacterium of the present invention can be obtained by introduction of the aforementioned DNAs into a bacterium inherently having the ability to produce L-amino acids. Alternatively, the bacterium of present invention can be obtained by imparting the ability to produce L-amino acid to the bacterium already harboring the DNAs.

As a parent strain which is to be enhanced in the activity of the protein of the present invention, the L-tryptophan-producing bacterium belonging to the genus Escherichia, the E. coli strains JP4735/pMU3028 (DSM10122) and JP6015/pMU91 (DSM10123) deficient in the tryptophanyl-tRNA synthetase encoded by mutant trpS gene (U.S. Pat. No. 5,756,345); E. coli strain SV164 (pGH5) having serA allele free from feedback inhibition by serine (U.S. Pat. No. 6,180,373); E. coli strains AGX17 (pGX44) (NRRL B-12263) and AGX6(pGX50)aroP (NRRL B-12264) deficient in the enzyme tryptophanase (U.S. Pat. No. 4,371,614); E. coli strain AGX17/pGX50, pACKG4-pps in which a phosphoenolpyruvate-producing ability is enhanced (WO9708333, U.S. Pat. No. 6,319,696) and the like may be used. The inventors of the present invention previously identified that the yddG gene encoding a membrane protein, which is not involved in biosynthetic pathway of any L-amino acid, conferred on a microorganism resistance to L-phenylalanine and several amino acid analogues when the wild-type allele of the gene was amplified on a multi-copy vector in the microorganism. Besides, the yddG gene can enhance production of L-phenylalanine or L-tryptophan when additional copies are introduced into the cells of the respective producing strain (Russian patent application 2002121670, WO03044192). Therefore, it is desired that the L-tryptophan-producing bacterium be further modified to have enhanced expression of yddG open reading frame.

Genes effective for L-tryptophan biosynthesis include genes of the trpEDCBA operon, genes of a common pathway for aromatic acids, such as aroF, aroG, aroH, aroB, aroD, aroE, aroK, aroL, aroA, and aroC genes, genes of L-serine biosynthesis, such as serA, serB, and serC genes, and the like.

As a parent strain which is to be enhanced in activity of the protein of the present invention, the phenylalanine-producing bacterium belonging to the genus Escherichia, the E. coli strain AJ12739 (tyrA::Tn10, tyrR); strain HW1089 (ATCC Accession No. 55371) harboring pheA34 gene (U.S. Pat. No. 5,354,672); mutant MWEC101-b strain (KR8903681); strains NRRL B-12141, NRRL B-12145, NRRL B-12146 and NRRL B-12147 (U.S. Pat. No. 4,407,952) and the like may be used. The phenylalanine-producing bacterium belonging to the genus Escherichia further includes the E. coli strain K-12 [W3110 (tyrA)/pPHAB], E. coli strain K-12 [W3110 (tyrA)/pPHAD], E. coli K-12 [W3110 (tyrA)/pPHATerm] and E. coli strain K-12 [W3110 (tyrA)/pBR-aroG4,pACMAB] named as AJ 12604 and the like (European patent EP488424B1).

As a parent strain which is to be enhanced in the activity of the protein of the present invention, the L-tyrosine-producing bacterium belonging to the genus Escherichia, the E. coli strains wherein a phosphoenolpyruvate-producing ability or the enzyme of common aromatic pathway is enhanced and the like may also be used (EP0877090A).

The method of the present invention includes a method for producing an L-amino acid, comprising the steps of cultivating the bacterium of the present invention in a culture medium, to allow the L-amino acid to be produced and accumulated in the culture medium, and collecting the L-amino acid from the culture medium. Also, the method of present invention includes a method for producing L-tryptophan comprising steps of cultivating the bacterium of the present invention in a culture medium, to allow L-tryptophan to be produced and accumulated in the culture medium, and collecting L-tryptophan from the culture medium. The method of present invention includes a method for producing L-phenylalanine comprising steps of cultivating the bacterium of the present invention in a culture medium, to allow L-phenylalanine to be produced and accumulated in the culture medium, and collecting L-phenylalanine from the culture medium. The method of present invention further includes a method for producing L-tyrosine, comprising steps of cultivating the bacterium of the present invention in a culture medium, to allow L-tyrosine to be produced and accumulated in the culture medium, and collecting L-tyrosine from the culture medium.

In the present invention, the cultivation, collection, and purification of L-amino acids, preferably aromatic amino acids such as L-tryptophan, L-phenylalanine, and L-tyrosine from the medium and the like may be performed in a manner similar to conventional fermentation methods wherein an amino acid is produced using a microorganism.

A medium used for culture may be either a synthetic or natural medium, so long as the medium includes a carbon source and a nitrogen source and minerals and, if necessary, appropriate amounts of nutrients which the microorganism requires for growth.

The carbon source includes various carbohydrates such as glucose and sucrose, and various organic acids. Depending on the mode of assimilation of the chosen microorganism, alcohol including ethanol and glycerol may be used.

As the nitrogen source, various ammonium salts such as ammonia and ammonium sulfate, other nitrogen compounds such as amines, a natural nitrogen source such as peptone, soybean-hydrolysate and digested fermentative microorganism may be used.

As minerals, potassium monophosphate, magnesium sulfate, sodium chloride, ferrous sulfate, manganese sulfate, calcium chloride, and the like may be used.

Additional nutrients can be added to the medium, if necessary. For instance, if the microorganism requires tyrosine for growth (tyrosine auxotrophy), a sufficient amount of tyrosine can be added to the medium for cultivation.

The cultivation is performed preferably under aerobic conditions such as a shaking culture, and stirring culture with aeration, at a temperature of 20 to 42° C., preferably 37 to 40° C. The pH of the culture is usually between 5 and 9, preferably between 6.5 and 7.2. The pH of the culture can be adjusted with ammonia, calcium carbonate, various acids, various bases, and buffers. Usually, a 1 to 5-day cultivation leads to the accumulation of the target L-amino acid in the liquid medium.

After cultivation, solids such as cells can be removed from the liquid medium by centrifugation or membrane filtration, and then the target L-amino acid can be collected and purified by ion-exchange, concentration and crystallization methods.

### EXAMPLES

The present invention will be explained more specifically below with reference to the following non-limiting Examples.

### Example 1

Identification of pgl gene from E. coli and nucleotide sequences comparison.

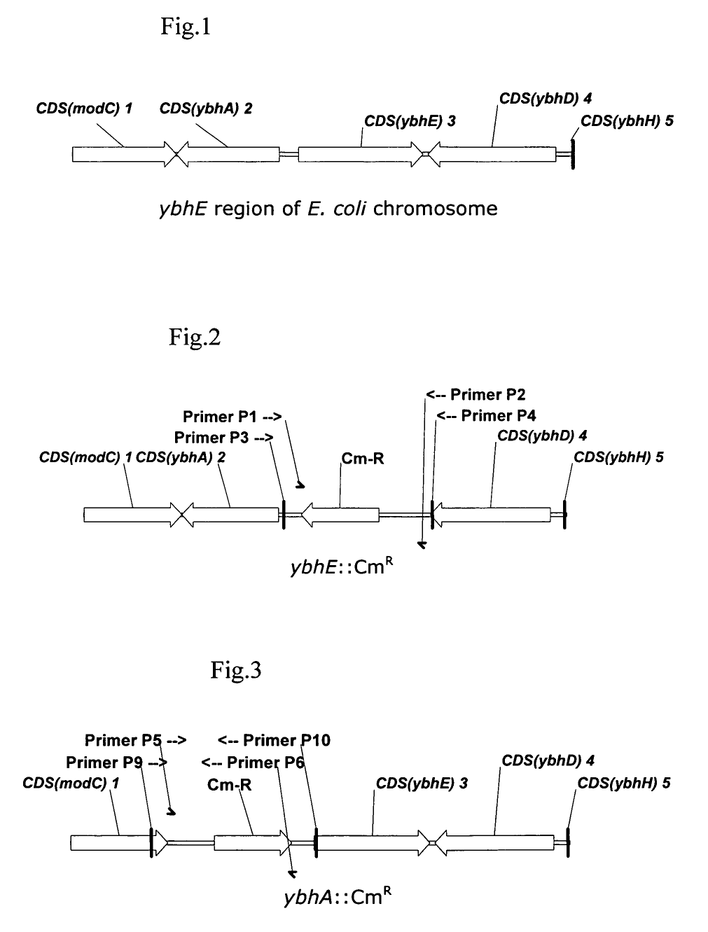

Kupor and Fraenkel mapped pgl mutation between chlD (presently known as modC) and bioA genes on the chromosome of E. coli (Kupor, S. R. and Fraenkel, D. G., J. Bacteriol., 100:3, 1296-1301 (1969)). This corresponds to 17.18 and 17.40 minutes of E. coli genetic map. In this region, there are eight open reading frames encoding proteins with unknown function. Further, E. coli Stock Center Database places pgl mutation between 17.20 and 17.22 minutes. These coordinates nearly exactly match the coordinates of the ybhE opened reading frame (ORF) located between ybhA and ybhD ORFs (FIG. 1).

BLAST searches performed with YbhE protein encoded by ybhE showed that there are many homologs with unknown function in different organisms, such as Shigella flexmeri (98.8% similarity), Salmonella typhi (92,8% similarity), Yersinia pestis (68,4% similarity), several homologs with known function, such as cytochrome D 1 heme domain from Bacillus anthracis (28% identity), 3-carboxymuconate cyclase from Pseudomonas fluorescens predicted by automated computational analysis (28% identity), muconate cycloisomerase from Trichosporon beigelii (26% identity), and the one from Bacillus cereus mentioned as 6-phosphogluconolactonase in the databanks under accession number NP_{—}833107, but without reference to published experimental work.

Also, three overlaid conserved protein domains were found using NCBI Conserved Domain Search. Two of them belong to conserved protein families with uncharacterized function, and one belongs to 3-carboxymuconate cyclase family.

A BLAST search within E. coli proteome did not revealed homologs of described 6-phosophogluconolactonases, for example, from Pseudomonas putida.

To identify whether the ORF marked as ybhE in the E. coli chromosome is pgl gene encoding 6-phosophogluconolactonase, consequent disruption of the ybhA, ybhE and ybhD ORFs was performed and the obtained mutants were checked for “maltose blue” phenotype (see below).

### Example 2

Disruption of the ybhE ORF. Substitution of the ybhE ORF by the DNA fragment carrying chloramphenicol resistance gene (Cm R).

To disrupt ybhE ORF, the DNA fragment carrying chloramphenicol resistance marker (Cm^{R}) encoded by cat gene was integrated into the chromosome of the E. coli strain BW25113 [pKD46] instead of the native ybhE ORF by method described by Datsenko K. A. and Wanner B. L. (Proc. Natl. Acad. Sci. USA, 2000, 97, 6640-6645) which is also called as a “Red-mediated integration” and/or “Red-driven integration”. The nucleotide sequence of the substituted native region of ybhE ORF and the amino acid sequence encoded by the ORF are presented in the Sequence listing (SEQ ID NOs: 1 and 2, respectively). Escherichia coli strain BW25113 containing the recombination plasmid pKD46 can be obtained from the E. coli Genetic Stock Center, Yale University, New Haven, USA, the accession number of which is CGSC7630.

A DNA fragment containing Cm^{R} marker was obtained by PCR using the commercially available plasmid pACYC184 (GenBank/EMBL accession number X06403, “Fermentas”, Lithuania) as the template and primers P1 (SEQ ID NO: 3) and P2 (SEQ ID NO: 4). Primer P1 contains 36 nucleotides homologous to the 5′-terminus of ybhE ORF and primer P2 contains 36 nucleotides homologous to 3′-terminus of ybhE ORF. These sequences of ybhE gene were introduced into primers P1 and P2 for further integration into the bacterial chromosome.

PCR was provided using the “TermoHybaid PCR Express” amplificator. The reaction mixture (total volume—50 μl) consisted of 5 μl of 10× PCR-buffer with 15 mM MgCl₂ (“Fermentas”, Lithuania), 200 μM each of dNTP, 25 pmol each of the exploited primers and 1 U of Taq-polymerase (“Fermentas”, Lithuania). Approximately 5 ng of the plasmid DNA was added in the reaction mixture as a template DNA for the PCR amplification. The temperature profile was the following: initial DNA denaturation for 5 min at 95° C., followed by 25 cycles of denaturation at 95° C. for 30 sec, annealing at 55° C. for 30 sec, elongation at 72° C. for 30 sec; and the final elongation for 7 min at 72° C.

Then, the amplified DNA fragment was purified by the agarose gel-electrophoresis, extracted using “GenElute Spin Columns” (“Sigma”, USA) and precipitated by ethanol. Nucleotide sequence of the constructed DNA fragment is presented in SEQ ID NO: 5.

The obtained DNA fragment purified as described above was used for electroporation and Red-mediated integration into the bacterial chromosome of the E. coli strain BW25113 [pKD46]. The recombinant plasmid pKD46 (Datsenko, K. A., Wanner, B. L., Proc. Natl. Acad. Sci. USA, 2000, 97, 6640-6645) with the thermosensitive replicon was used as the donor of the phage λ-derived genes responsible for functioning in the Red-mediated recombination system.

BW25113[pKD46] cells were grown overnight at 30° C. in the liquid LB-medium with addition of ampicillin (100 μg/ml), then diluted 1:100 by the SOB-medium (Yeast extract, 5 g/l; NaCl, 0.5 g/l; Tryptone, 20 g/l; KCl, 2.5 mM; MgCl₂, 10 mM) with addition of ampicillin (100 μg/ml) and L-arabinose (10 mM) (arabinose is used for inducing the plasmid encoding genes of Red system) and grown at 30° C. to reach the optical density of the bacterial culture OD₆₀₀=0.4-0.7. The grown cells from 10 ml of the bacterial culture were washed 3 times by the ice-cold de-ionized water, followed by suspending in 100 μl of the water. 10 μl of DNA fragment (100 ng) dissolved in the de-ionized water was added to the cell suspension. The electroporation was performed by “Bio-Rad” electroporator (USA) (No. 165-2098, version 2-89) according to the manufacturer's instructions. Shocked cells were added to 1-ml of SOC medium (Sambrook et al, “Molecular Cloning A Laboratory Manual, Second Edition”, Cold Spring Harbor Laboratory Press (1989)), incubated 2 hours at 37° C., and then were spread onto L-agar containing 25 μg/ml of chloramphenicol. Colonies grown within 24 h were tested for the presence of Cm^{R} marker instead of native ybhE ORF by PCR using primers P3 (SEQ ID NO: 6) and P4 (SEQ ID NO: 7). For this purpose, a freshly isolated colony was suspended in 20 μl water and then 1 μl of obtained suspension was used for PCR. Temperature profile was the following: initial DNA denaturation for 10 min at 95° C.; then 30 cycles of denaturation at 95° C. for 30 sec, annealing at 55° C. for 30 sec and elongation at 72° C. for 1 min; the final elongation for 7 min at 72° C. A few Cm^{R} colonies tested contained the desired 1279 bp DNA fragment, confirming the presence of Cm^{R} marker DNA instead of the native ybhE ORF. One of the obtained strains was cured from the thermosensitive plasmid pKD46 by culturing at 37° C. and the resulting strain was named E. coli strain BW25113-ΔybhE.

The structure of the bacterial DNA region with disrupted ybhE ORF is shown in FIG. 2.

### Example 3

Disruption of the ybhA and ybhD ORFs. Substitution of the ybhA and ybhD ORFs by the DNA fragments carrying chloramphenicol resistance gene (Cm^{R}).

To disrupt ybhA and ybhD ORFs, the DNA fragments carrying chloramphenicol resistance marker (Cm^{R}) encoded by cat gene were separately integrated in the chromosomes of the E. coli strain BW25113 [pKD46] instead of the native ybhA and ybhD ORFs by the method described in Example 2.

To obtain the fragments for electroporation and disruption of ybhA and ybhD ORFs, two pairs of primers P5 (SEQ ID NO: 8) and P6 (SEQ ID NO: 9), and P7 (SEQ ID NO: 10) and P8 (SEQ ID NO: 11), respectively, were synthesized and used for PCR. Primer P5 contains 36 nucleotides homologous to 3′-terminus of ybhA ORF. Primer P6 contains 36 nucleotides homologous to 5′-terminus of ybhA ORF. Primer P7 contains 36 nucleotides complementary to the 3′-terminus of ybhD ORF. And primer P8 contains 36 nucleotides complementary to 5′-terminus of ybhD ORF. These sequences were introduced into primers P5, P6, P7 and P8 for further integration into the bacterial chromosome.

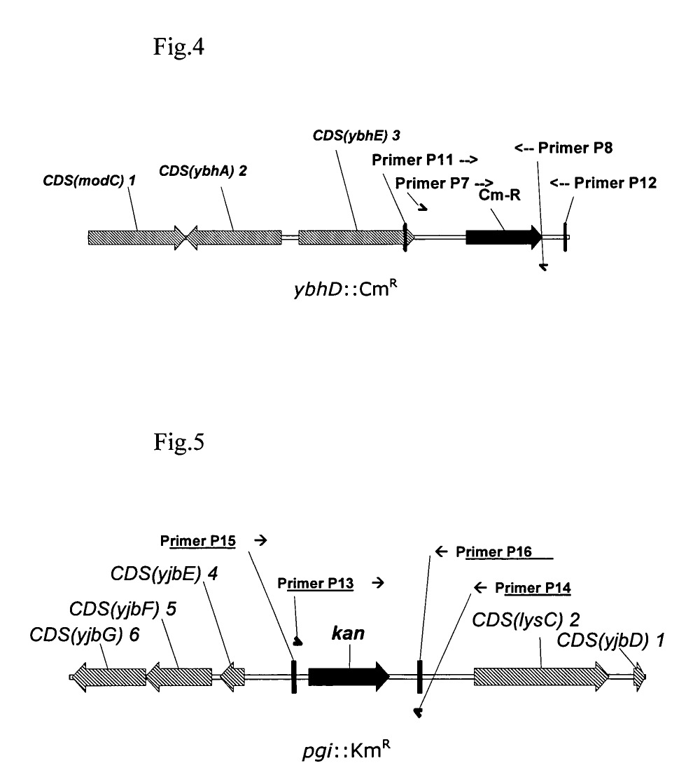

Nucleotide sequences of the constructed DNA fragments are presented in SEQ ID NO: 12 and SEQ ID NO: 13, respectively. Nucleotide sequences of the substituted native regions of ybhA and ybhD ORFs are presented in GenBank under accession number NC_{—}000913.1 (nucleotide numbers 796836 to 797654 and 798845 to 799777, gi: 16128734 and gi:33347481, respectively). The structure of the bacterial DNA regions with disrupted ybhA and ybhD ORFs are shown in FIG. 3 and FIG. 4, respectively.

After electroporation, corresponding colonies were tested for the presence of Cm^{R} marker by PCR using primers P9 (SEQ ID NO: 14) and P10 (SEQ ID NO: 15) for disruption of ybhA ORF and primers P11 (SEQ ID NO: 16) and P12 (SEQ ID NO: 17) for disruption of ybhD ORF.

In the first case, a few Cm^{R} colonies tested contained the desired 1424 bp DNA fragment, confirming the presence of Cm^{R} gene instead the native ybhA ORF. In the second case, a few Cm^{R} colonies tested contained the desired 1386 bp DNA fragment, confirming the presence of Cm^{R} gene instead the native ybhD ORF. In each case, one of the obtained strains was cured from thermosensitive plasmid pKD46 by culturing at 37° C. and the resulting strains were designated E. coli strain BW25113-ΔybhA and BW25113-ΔybhD, respectively.

### Example 4

Checking the ybhE⁻, ybhA⁻ and ybhD⁻ mutants for “maltose blue” phenotype.

Each of three obtained mutant strains was tested for “maltose blue” phenotype by the method described by Kupor, S. R. and Fraenkel, D. G. (J. Bacteriol., 100:3, 1296-1301 (1969)). The cultures were patched on plates with M9 minimal media containing 0.8% of maltose. After 6 hours, incubation plates were flooded with 5 ml of solution containing 0.01M I₂ and 0.03M KI, and the patch color was visually scored as “blue” or “not blue”.

The obtained strain BW25113-ΔybhE was scored as “blue”, while strains BW25113-ΔybhA, BW25113-ΔybhD and BW25113 (as control strain) were “not blue”.

### Example 5

Construction of the double mutant strains carrying pgi and ybhE or ybhD deletions. Comparison of growth of such strains on different carbon sources.

The mutant strain lacking phosphoglucose isomerase (pgi⁻) grew slowly on glucose using the oxidative branch of pentose phosphate pathway exclusively. A secondary mutant also lacking phosphogluconolactonase (pgl) catalyzing the second step of this branch should grow more slowly due to the spontaneous hydrolysis of 6-phosphogluconolactone to gluconate-6-phosphate only. So, if ybhE ORF is really pgl gene, the double pgi, ybhE mutant will grow slower than wild-type strain and pgi mutant. To support this suggestion, double pgi, ybhE mutant was prepared.

The mutation in pgi gene was performed by substitution of the native bacterial chromosome region in E. coli strain BW25113 [pKD46] with the DNA fragment carrying kanamycin resistance gene (Km^{R}) by the method described in Example 2. The nucleotide sequence of the substituted native region of pgi gene is presented in GenBank under accession number NC_{—}000913.1 (nucleotide numbers 4231337 to 4232986; gi:16131851).

DNA fragments carrying the Km^{R} gene were obtained by PCR using the commercially available plasmid pUC4KAN (GenBank/EMBL accession number X06404, “Fermentas”, Lithuania) as the template and primers P13 (SEQ ID NO: 18) and P14 (SEQ ID NO: 19). Primer P13 contains 36 nucleotides homologous to the 3′-terminus of pgi gene and primer P14 contains 36 nucleotides of homologous to the 5′-terminus of pgi gene. These sequences from the pgi gene were introduced into primers P13 and P14 for further integration into the bacterial chromosome.

PCR was conducted as described in Example 2.

Then, the amplified DNA fragment was concentrated by agarose gel-electrophoresis, extracted from the gel by the centrifugation through “GenElute Spin Columns” (“Sigma”, USA) and precipitated by ethanol. The nucleotide sequence of the constructed DNA region is presented in SEQ ID NO: 20.

The obtained DNA fragment purified as described above was used for electroporation and Red-mediated integration into the bacterial chromosome of the E. coli strain BW25113[pKD46] as described in Example 2, except that cells were spread after electroporation onto L-agar containing 50 μg/ml of kanamycin.

Colonies grown within 24 h were tested for the presence of Km^{R} marker instead of pgi gene by PCR using primers P15 (SEQ ID NO: 21) and P16 (SEQ ID NO: 22). For this purpose, a freshly isolated colony was suspended in 20 μl water and then 1 μl of obtained suspension was used for PCR. PCR conditions were as described in Example 2. A few Km^{R} colonies tested contained the desired 1286 bp DNA fragment confirming the presence of Km^{R} gene instead of pgi gene. One of the obtained strains was cured from thermosensitive plasmid pKD46 by culturing at 37° C. and the resulting strain was named E. coli strain BW25113-Δpgi.

The structure of the bacterial DNA region with the pgi gene deleted is shown on FIG. 5.

The pgi deletion was transduced by the method of Fraenkel (J. Bacteriol. 93(1967), 1582-1587) into E. coli MG1655 strain followed by selection on plates containing kanamycin. The obtained strain was named MG-Δpgi. Then, mutations in ybhE and ybhD ORFs were transduced from strains BW25113-ΔybhE and BW25113-ΔybhD described in Examples 2 and 3 into the obtained strain, followed by selection on plates containing chloramphenicol. Obtained strains were designated MG-Δpgi-ΔybhE and MG-Δpgi-ΔybhD, respectively.

These two strains, together with MG1655 and MG1655-Δpgi, were patched on M9 minimal plates with glucose or gluconate as a carbon source. After 24 h of incubation the growth of the strains was visually tested. The growth of MG-Δpgi-ΔybhE was worse than for all other strains on the plate with glucose and indistinguishable on the plate with gluconate.

### Example 6

Construction of the plasmid carrying pgl gene from Pseudomonas putida and complementation of the ybhE mutation.

The pgl genes from several organisms have been described. Among them is the 6-phosphogluconolactonase from Pseudomonas putida, an organism rather closely related to E. coli. Several other genes were cloned from P. putida into E. coli and complementation of the corresponding mutations presented in E. coli was reported (Ramos-Gonzalez, M. I. and Molin, S., J. Bacteriol., v180, 13, p. 3421, 1998).

The pgl gene from P. putida was cloned using the primers 17 (SEQ ID No. 23) and 18 (SEQ ID No. 24). The primer P17 contains a sequence which is identical to a sequence from 1 to 19 bp of the pgl gene from P. putida. The primer also contains the ribosome binding site (RBS) of lacZ gene from E. coli located upstream and a recognition site for the restriction enzyme SacI introduced at the 5′-end thereof. The primer P18 contains a sequence complementary to a sequence from 709 to 729 bp of the pgl gene from P. putida and a recognition site for restriction enzyme EcoRI introduced at the 5′-end thereof.

The chromosomal DNA of P. putida KT2440 strain TG1 (Bagdasarian, M. & Timmis, K. N. In Current Topics of Microbiology and Immunology, eds. Goebel, W. & Hofschneider, P. H. (Springer, Berlin), pp. 47-67 (1981)) was prepared by a typical method. PCR was carried out on “Perkin Elmer GeneAmp PCR System 2400” under the following conditions: 40 sec. at 95° C., 40 sec. at 53° C., 40 sec. at 72° C., 25 cycles with Taq polymerase (Fermentas). The obtained PCR fragment containing the pgl gene from P. putida with RBS of lacZ gene was treated with SacI and EcoRI restrictases and inserted into multicopy vector pUC19 previously treated with the same restrictases. Thus, the plasmid pUC19-pgl was obtained.

Strain BW25113-ΔybhE was transformed by the obtained plasmid pUC19-pgl. The culture was patched on minimal-maltose plates containing 100 μg/ml of ampicillin and treated as described above to check the “maltose blue” phenotype. Transformants did not show the “maltose blue” phenotype in the contrast to the control strain BW25113-ΔybhE.

So, the cloned copy of pgl gene from Pseudomonas putida complements the ybhE mutation in E. coli once more supporting our hypothesis that ybhE ORF is the coding region of pgl gene.

### Example 7

Measuring the 6-phosphogluconolactonase activity in ybhE mutant.

The overnight cultures of strains BW25113 and BW25113-ΔybhE were diluted 50 times with minimal M9 media containing glucose. Cells were grown until the optical density of the culture reached OD₅₄₀=1. Extracts were prepared from 3 ml cultures. Cells were washed by physiological solution, resuspended in 400 μl of potassium phosphate buffer (pH 7.0) and sonicated. Then, the supernatant fractions obtained after centrifugation were used in the assay without further dilution.

For the measurement of 6-phosphogluconolactonase activity, the method described by Collard, F. et al. (FEBS Letters 459 (1999) 223-226) was used. Lactone was prepared extemporaneously by incubating 50 μM glucose-6-phosphate (Sigma, USA) in the presence of 0.2 mM NADP, 25 mM HEPES (pH 7.1), 2 mM MgCl₂ and 1.75 U yeast glucose-6-phosphate dehydrogenase (Sigma, USA) at 30° C. (total volume−1 ml). When the optical density of the reaction mixture at A₃₄₀ reached a plateau, 0.5 U/ml 6-phosphogluconate dehydrogenase (Sigma, USA) along with earlier obtained supernatant fractions to be assayed were added and optical density at A₃₄₀ was further measured for about 10 min. The amount of protein was measured according to method of Bradford, M. M. (Anal. Biochem. 72, 248-254 (1976)). The data obtained are shown in Table 1. Activity is shown in relative units per mg of total protein.

TABLE 1

| | 6-phosphogluconolactonase activity |
|---|---|
| Strain | (for various extract concentrations) |
| BW25113 | 4.0 |
| | 6.1 |
| | 5.4 |
| BW25113-ΔybhE | 0.3 |
| | 0.2 |
| Spontaneous hydrolysis | 0.3 |

As could be seen from the table, 6-phosphogluconolactonase activity in ybhE mutant is at least one order of magnitude less than in the “wild-type” strain and is comparable with rate of spontaneous hydrolysis.

### Example 8

Deletion of the zwf-edd-eda operon. Substitution of the zwf-edd-eda genes region by the DNA fragments carrying kanamycin resistance gene (Km R).

In order to obtain the strain with increased YbhE expression we planned the integration of constitutive promoter derived from P_{tac} between ybhE RBS and its native promoter using Red-mediated integration (see Example 9).

But we failed to provide such chromosome modification of the “wild-type” strain MG1655. We can not explain the toxic effect of enhanced expression of pgl(ybhE), but we proposed that it is concerned with increased 6-phosphogluconolactonase activity leading to disbalance of Pentose-Phosphate Pathway (PPP) and possible accumulation of some toxic intermediates (or starvation of some essential for cell survival ones). So, we decided to completely turn off the PPP by deletion of zwf gene encoding the first enzyme of this pathway.

The deletion of zwf-edd-eda operon was performed by the method described in Example 5 for pgi gene. The nucleotide sequence of the substituted native regions of zwf-edd-eda operon is presented in GenBank under accession number NC_{—}000913.1 (nucleotide numbers 1932863 to 1934338, gi:16129805; 1930817 to 1932628, gi:16129804 and 1930139 to 1930780, gi:16129803 for zwf, edd and eda genes, respectively). DNA fragments carrying Km^{R} gene were obtained by PCR using primers P19 (SEQ ID NO: 25) and P20 (SEQ ID NO: 26). Primer P19 contains 36 nucleotides complementary to the 3′-terminus of eda gene. Primer P20 contains 36 nucleotides complementary to the 5′-terminus of zwf gene. The nucleotide sequence of the constructed DNA fragment is presented in SEQ ID NO: 27.

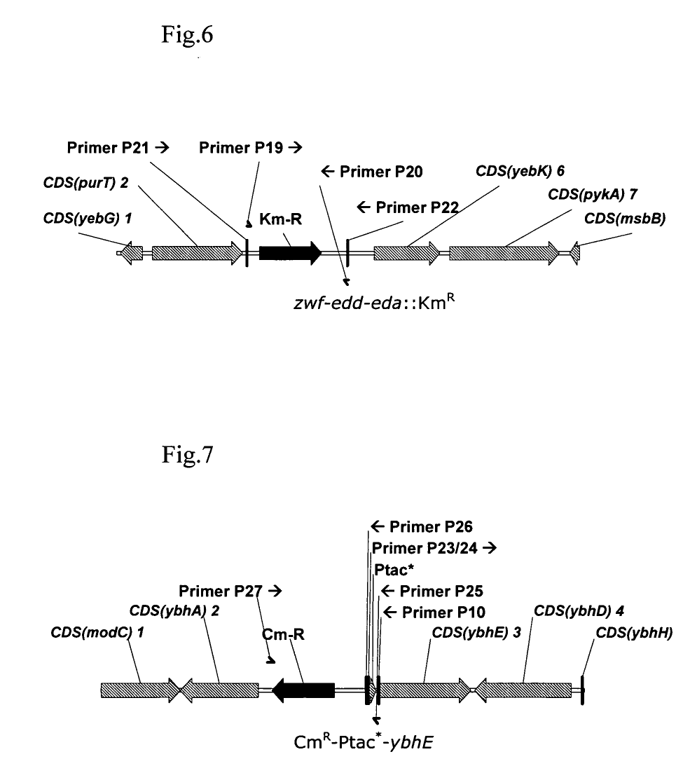

Colonies grown within 24 h were tested for the presence of the Km^{R} marker instead of the zwf-edd-eda operon by PCR using primers P21 (SEQ ID NO: 28) and P22 (SEQ ID NO: 29). A few Km^{R} colonies tested contained the desired 1287 bp DNA fragment, confirming the presence of the Km^{R} gene instead of the zwf-edd-eda operon. One of the obtained strains was cured from the thermosensitive plasmid pKD46 by culturing at 37° C. and the resulting strain was designated E. coli strain BW25113-Δzwf-edd-eda. The structure of the bacterial DNA region having the zwf-edd-eda operon deleted is shown in FIG. 6.

### Example 9

Substitution of the native upstream region of ybhE gene located on the E. coli chromosome with a novel regulatory element carrying the synthetic P_{tac*}-promoter.

For further integration of artificial P_{tac*} promoters of various strength upstream of pgl (ybhE) gene, retransformation of E. coli strain BW25113-Δzwf-edd-eda with pKD46 plasmid was performed. The obtained kanamycin and ampicillin resistant strain was designated E. coli strain BW25113-Δzwf-edd-eda[pKD46]. Since the pKD46 plasmid is thermosensitive, further selection of transformants was carried out at 30° C.

It is a well-established fact that mutants with modified “−35”-region of the promoter, which is recognized by the complex of E. coli RNA polymerase with σ⁷⁰, possess significantly changed efficiency of the transcription initiation (WO00/18935). So, among the obtained promoters created on the initial random promoter-like sequence, the promoters with different strength might be obtained. Thus, this general approach could be exploited for fine-tuning of the expression level of the gene of interest. The inventors of the present invention previously obtained the library of modified P_{tac} promoters with different strengths (hereinafter such modified P_{tac} promoters are marked with an asterisk). These promoters differ in 4 central nucleotides of “−35” region. In the present work, two P_{tac*} promoters with different strengths were used. Based on the values of the activity of β-galactosidase expressed under the control of the corresponding promoter, those were named as P_{tac-10000} (usual P_{tac}) and P_{tac-3900} (with TTGC central nucleotides instead of original TGAC).

Then, each of these artificial P_{tac*} promoters were integrated upstream of the coding region of the pgl gene into the chromosome of the E. coli strain BW25113-Δzwf-edd-eda[pKD46] by the method described above (see Example 2). In addition, the artificial DNA fragment which has the chloramphenicol resistance gene (Cm^{R}) upstream of the promoter region was integrated (see FIG. 7).

Construction of the above-mentioned artificial DNA fragments integrated into the corresponding region of the bacterial chromosomes was fulfilled in the several steps. For the first step, a DNA fragment, which carried the BglII restriction site in the upstream region and corresponding P_{tac*} promoter was obtained by PCR.

The chromosomal DNAs from E. coli MG1655 strains having artificial P_{tac-3900} promoter and P_{tac-10000} promoter integrated into chromosome were used for the PCR as a templates. PCR was provided using primers P23 (SEQ ID NO: 30) and P24 (SEQ ID NO: 31) in case of P_{tac-3000} and P_{tac-10000}, respectively, and primer P25 (SEQ ID NO: 32) in both cases. Primers P23 and P24 contain BglII—restriction site introduced in the 5′-end thereof. Primer P25 contains 11 nucleotides (including RBS) upstream of pgl gene and the first 25 nucleotides of pgl coding region. The above-mentioned sequences were introduced into primer P25 for further integration into the bacterial chromosome.

PCR was conducted using the amplificatory “TermoHybaid PCR Express PCR System”. The reaction mixture (total volume 50 μl) consists of: 5 μl of 10× PCR-buffer with 15 mM MgCl₂ (“Fermentas”, Lithuania), 200 μM each of dNTP, 25 pmol each of the exploited primers and 1 u Taq-polymerase (“Fermentas”, Lithuania). 0.5 μg of the chromosomal DNA was added in the reaction mixture as a template DNA for the further PCR-driven amplification. The temperature PCR condition were as follows: initial DNA denaturation for 5 min at 95° C. followed by 25 cycles of denaturation at 95° C. for 30 sec, annealing at 53° C. for 30 sec, elongation at 72° C. for 30 sec and the final polymerization for 7 min at 72° C.

The second stage of construction of the DNA fragment of interest was performed. Cm^{R} gene was amplified by PCR using the commercially available plasmid pACYC184 (GenBank/EMBL accession number X06403, “Fermentas”, Lithuania) as the template and primers P26 (SEQ ID NO: 33) and P27 (SEQ ID NO: 34). Primer P26 contains the BglII-restriction site used for further joining with the earlier obtained DNA fragment carrying P_{tac*} promoter. Primer P27 contains 46 nucleotides complementary to nucleotides 58 to 12 located upstream of pgl (ybhE) gene start codon from E. coli, necessary for further integration of the fragment into the bacterial chromosome.

Amplified DNA fragments were then concentrated by agarose gel-electrophoresis, extracted from the gel by centrifugation through “GenElute Spin Columns” (“Sigma”, USA) and precipitated by ethanol. Then, the two obtained DNA fragments were treated with BglII restriction endonuclease followed by ligation using T4 DNA ligase (Maniatis T., Fritsch E. F., Sambrook, J.: Molecular Cloning: A Laboratory Manual. 2^{nd} edn. Cold Spring Harbor, N.Y.: Cold Spring Harbor Laboratory Press, 1989).

The ligated product was amplified by PCR using primers P25 and P27. PCR was conducted with a reaction mixture (total volume−50 μl) consisting of: 5 μl of 10× AccuTaq LA buffer (“Sigma”, USA), 200 μM each of dNTP, 25 pmol each of the exploited primers and 1μ AccuTaq LA polymerase (“Sigma”, USA). Approximately 50 ng of ligated DNA product was added in the reaction mixture as a template. The PCR temperature cycles were as follows: initial DNA denaturation for 5 min at 95° C. followed by 25 cycles of denaturation at 95° C. for 30 sec, annealing at 55° C. for 30 sec, elongation at 72° C. for 4 min and the final polymerization for 7 min at 72° C.

Nucleotide sequences of the constructed DNA regions are presented in SEQ ID NO: 35 and 36 for P_{tac-3900} and P_{tac-10000} promoters, respectively.

The obtained DNA fragments purified as described above were used for electroporation and Red-mediated integration into the bacterial chromosome of the E. coli strain BW25113Δzwf-edd-eda[pKD46] as described in Example 2.

Colonies which grew within 24 h in the medium with chloramphenicol were tested for the presence of Cm^{R} marker upstream of the pgl gene by PCR using primers P27 (SEQ ID NO: 34) and P10 (SEQ ID NO: 15). The same colonies were also tested for the presence of P_{tac*} promoter region upstream of the pgl gene by PCR using primers P23 (SEQ ID NO: 30) and P24 (SEQ ID NO: 31) for P_{tac-3900} and P_{tac-10000}, respectively, and P10 (SEQ ID NO: 15). For this purpose, a freshly isolated colony was suspended in 20 μl water and then 1 μl of the suspension was used for PCR. PCR conditions were as follows: initial DNA denaturation for 10 min at 95° C.; then 30 cycles of denaturation at 95° C. for 30 sec, annealing at 54° C. for 30 sec and elongation at 72° C. for 1 min; the final polymerization for 7 min at 72° C. A few Cm^{R} colonies tested contained the desired 1193 bp and 124 bp DNA fragments confirming the presence of whole constructed DNA regions upstream of pgl gene and hybrid regulatory element, carrying the P_{tac*} promoter in E. coli chromosomes, respectively. In both cases, one of the obtained strains was cured from thermosensitive plasmid pKD46 by culturing at 37° C. and resulting strains were named as E. coli strain BW25113-Ptac-3900-ybhE and BW25113-Ptac-10000-ybhE, respectively. The structure of constructed DNA region upstream of the pgl gene is shown on FIG. 7.

### Example 10

Measuring the 6-phosphogluconolactonase activity in strains with enhanced expression of pgl gene.

The activity of 6-phosphogluconolactonase from strains BW25113-P_{tac-3900}-ybhE and BW25113-P_{tac-10000}-ybhE was measured as described in Example 7. Data obtained are shown in Table 2. The level of spontaneous hydrolysis has been subtracted.

TABLE 2

| | 6-phosphogluconolactonase activity, |
|---|---|
| Strain | relative units |
| BW25113 | 5.6 |
| BW25113-P_{tac-3900}-ybhE | 21.1 |
| BW25113-P_{tac-10000}-ybhE | 54.0 |

So, the enhanced expression of pgl gene leads to an increase in the 6-phosphogluconolactonase activity.

### Example 11

Effect of enhanced expression of pgl gene on tryptophan production.

The tryptophan-producing E. coli strain SV164[pMW-P_{lacUV5}-serA5-fruR, pYDDG2] was used as a parental strain for evaluation of effect of enhanced pgl gene expression on tryptophan production. The strain SV164 is described in detail in U.S. Pat. No. 6,180,373. The strain SV164[pMW-P_{lacUV5}-serA5-fruR, pYDDG2] is a derivative of the strain SV164, and additionally containing plasmids pMW-P_{lacUV5}-serA5-fruR and pYDDG2. Plasmid pMW-P_{lacUV5}-serA5-fruR carries a mutant serA5 gene encoding the protein, which is free from feedback inhibition by serine (WO2004090125 A2). Amplification of the serA5 gene is necessary for increasing the amount of serine, which is the precursor of L-tryptophan (U.S. Pat. No. 6,180,373). Plasmid pYDDG2 is constructed on the basis of pAYCTER3 vector (WO03/044192) and contains the yddG gene encoding transmembrane protein (putative exporter) useful for L-tryptophan production. The pAYCTER3 vector is a derivative of a pAYC32, which is a moderate copy number and very stable vector constructed on the basis of plasmid RSF1010, and harboring a marker for streptomycin resistance (Christoserdov A. Y., Tsygankov Y. D, Broad-host range vectors derived from a RSF 1010 Tnl plasmid, Plasmid, 1986, v. 16, pp. 161-167). The pAYCTER3 vector was obtained by introduction of the polylinker from pUC19 plasmid and the strong terminator rrnB into pAYC32 plasmid instead of its promoter.

To test the effect on tryptophan production of enhanced expression of pgl gene which is under the control of P_{tac*} promoters, the DNA fragments from the chromosome of the above-mentioned E. coli strains BW25113-P_{tac-3900}-ybhE and BW25113-P_{tac-10000}-ybhE were transferred to a tryptophan-producing E. coli strain SV164 [pMW-P_{lacUV5}-serA5-fruR] by P1 transduction (Miller, J. H. (1972) Experiments in Molecular Genetics, Cold Spring Harbor Lab. Press, Plainview, N.Y.). Then, plasmid pYDDG2 was introduced into both the SV164[pMW-P_{lacUV5}-serA5-fruR] strain and the resulted transductants.

The SV164 [pMW-P_{lacUV5}-serA5-fruR, pYDDG2], SV164-P_{tac-3900-ybhE}[pMW-P_{lacUV5}-serA5-fruR, pYDDG2] and SV164-P_{tac-10000}-ybhE[pMW-P_{lacUV5}-serA5-fruR, pYDDG2] strains were cultivated overnight with shaking at 37° C. in 3 ml of nutrient broth supplemented with 100 μg/ml of ampicillin and 50 μg/ml of streptomycin. 0.3 ml of the obtained cultures were inoculated into 3 ml of a fermentation medium containing said antibiotics in 20×200 mm test tubes, and cultivated at 37° C. for 40 hours with a rotary shaker at 250 rpm.

The composition of the fermentation medium is presented in Table 3.

TABLE 3

| Sections | Component | Final concentration | |
|---|---|---|---|
| A | Glucose/sucrose | 40 | g/L |
| | MgSO₄.7H₂O | 0.3 | g/L |
| | MnSO₄.5H₂O | 5 | mg/L |
| | Mameno | 0,058 | g/L of Total Nitrogen |
| | (NH₄)₂SO₄ | 15 | g/L |
| | KH₂PO₄ | 0,268 | g/L |
| | NaCl | 0.143 | g/L |
| | L-Methionine | 0,086 | g/L |
| | L-Phenylalanine | 0,286 | g/L |
| | L-Tyrosine | 0,286 | g/L |
| | KCl | 0,286 | g/L |
| | FeSO₄.7H₂O | 5 | mg/L |
| | Sodium citrate | 667 | mg/L |
| | CaCl₂.2H₂O | 4,29 | mg/L |
| Sterilize at 116° C. for 30 min. | | | |
| B | Thiamine HCl | 2,5 | mg/L |
| | Na₂MoO₄.2H₂O | 0,15 | mg/L |
| | H₃BO₃ | 2,5 | mg/L |
| | CoCl₂.6H₂O | 0,7 | mg/L |
| | CuSO₄.5H₂O | 0,25 | mg/L |
| | ZnSO₄.7H₂O | 0,3 | mg/L |
| Sterilize at 110° C. for 30 min. | | | |
| C | Pyridoxin | 45 | mg/L |
| Filtration | | | |

Section A had pH 7.1 adjusted by NH₄OH. Each section was sterilized separately.

After the cultivation, the amount of L-tryptophan which accumulated in the medium was determined by TLC. 10×15 cm TLC plates coated with 0.11 mm layers of Sorbfil silica gel without fluorescent indicator (Stock Company Sorbpolymer, Krasnodar, Russia) were used. Sorbfil plates were developed with a mobile phase: propan-2-ol:ethylacetate:25% aqueous ammonia:water=16:16:3:9 (v/v). A solution (2%) of ninhydrin in acetone was used as a visualizing reagent. Obtained data are presented in the Table 4.

TABLE 4

| | | Amount of tryptophan, |
|---|---|---|
| Strain | OD₆₀₀ | g/l |
| SV164[pMW-P_{lacUV5}-serA5-fruR, pYDDG2] | 7.5 | 4.20 |
| SV164-P_{tac-3900-ybhE}[pMW-P_{lacUV5}-serA5-fruR, pYDDG2] | 7.5 | 4.61 |
| SV164-P_{tac-10000-ybhE}[pMW-P_{lacUV5}-serA5-fruR, pYDDG2] | 7.5 | 4.92 |

As it can be seen from Table 4, the enhancement of pgl gene expression improved tryptophan productivity of the SV164[pMW-P_{lacUV5}-serA5-fruR, pYDDG2] strain.

### Example 12

Purification of His-tagged YbhE protein and determination of its 6-PGL (6-phosphogluconolactonase) activity.

All results previously described in Examples 1-7 serve as indirect indications that ybhE ORF is the pgl gene that encodes the functionally active 6-PGL in E. coli. On the other hand, it is possible that the ybhE ORF encodes, for instance, a positive regulator of expression of another unknown gene that, in turn, encodes 6-PGL. So, a final conclusion concerning the nature of ybhE ORF can be made only by direct determination of the biological activity of its protein product.

For this purpose, ybhE ORF was overexpressed by exploiting the T7 expression system, including E. coli BL21(DE3) as the recipient strain carrying T7 RNA polymerase gene in the chromosome, and pET-22b(+) vector plasmid with T7 late promoter and efficient RBS of T7 gene 10. For the convenience of the following protein purification 6 His codons were inserted at 5′-end of ybhE ORF, exactly after ATG initiation codon.

To clonethe ybhE ORF with a His-Tag sequence in the T7 expression system, PCR was conducted with primers P28 (SEQ ID NO: 37) and P29 (SEQ ID NO: 38) using chromosomal DNA of the E. coli strain MG1655 as the template. Primer P28 contains a NdeI restriction site, and an ATG-codon linked to 6 additional histidine codons before the second codon of the ybhE ORF. Primer P29 contains a BamHI restriction site at its 5′-end for further cloning. The amplified DNA fragment was isolated, treated with NdeI and BamHI restrictases, and ligated into a pET-22b(+) plasmid which had been treated with the same restrictases. Construction of the obtained pET-HT-ybhE plasmid was verified by sequencing.

Then, BL21 (DE3) cells carrying the T7 RNA polymerase gene under the control of a lactose promoter in their chromosome were transformed with a pET-HT-ybhE plasmid. The overnight culture from a single colony was diluted 50 times with LB and grown to OD₆₀₀˜1.0 followed by addition of IPTG (1 mM) for inducing of T7 RNA polymerase-driven expression of ybhE ORF in the recombinant plasmid. After 2 hours of incubation, cells were collected from 20 ml. Cell extracts were prepared by sonication in the buffer containing 20 mM Tris-HCl, pH 8.0 and 2 mM PMSF. Then, probes were centrifuged for 20 min at 16,000×g and 4° C. followed by purification of His-tagged protein from the supernatant using HiTrap Chelating HP Columns (Amersham Bioscience) as recommended by producer.

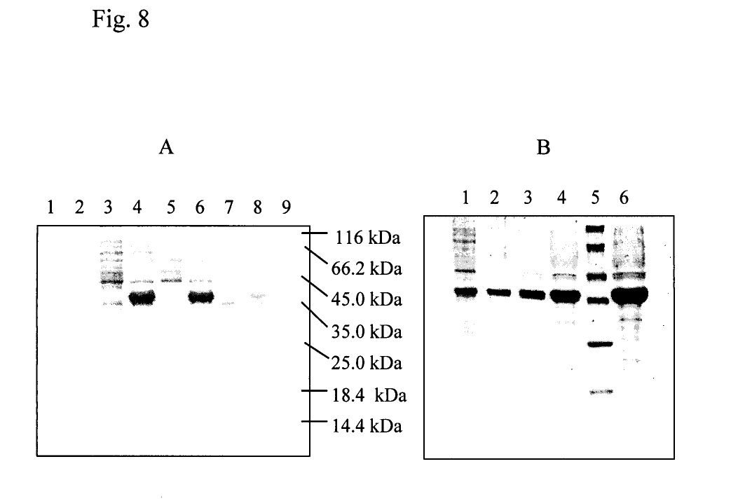

Two hours after induction of the T7 expression system in logarithmic culture, the accumulation of the protein with the electrophoretic mobility corresponding to His-tagged YbhE (protein with MW>>37 KDa) was observed. The amount of the protein was about 15% of total cellular polypeptides. The protein was observed mostly in a soluble phase (see, FIG. 8A).

The obtained His₆-YbhE protein was purified using a Ni-NTA column. Determination of the level of the recombinant protein synthesis and control of the purification process were provided by SDS-PAGE electrophoresis according to method described by Laemmli U. K. (Nature, 227, 680-685 (1970)). As can be seen in FIG. 8B, the purity of the obtained protein is more than 90%, and it exhibited 6-PGL activity in the standard lactonase test (Collard, F. et al, FEBS Letters, 459, 223-226 (1999)); Example 7). It is interesting to note that the determined specific 6-PGL activity for the purified His₆-YbhE (780 U/mg) is very close to the earlier reported activity of the similarly His₆-tagged human 6-PGL (710 U/mg) (Collard, F. et al, FEBS Letters, 459, 223-226 (1999)).

Thus, it can be concluded that the ybhE ORF from E. coli with unknown function is indeed the pgl gene encoding 6-PGL.

### Example 13

Effect of enhanced expression of pgl gene on phenylalanine production.

The phenylalanine-producing E. coli strain AJ12739 was used as a parental strain for evaluating the effect of enhanced pgl gene expression on tryptophan production. The strain AJ12739 was deposited in the Russian National Collection of Industrial Microorganisms (VKPM) (Russia, 113545 Moscow, 1^{st} Dorozhny proezd, 1) on Nov. 6, 2001 under accession number VKPM B-8197.

Chromosomal DNA fragments from BW25113-P_{tac-3900}-ybhE and BW25113-P_{tac-1000}-ybhE strains were transfered into the phenylalanine-producing strain AJ12739 by P1 transduction, resulting in the AJ12739 P_{tac-3900}-ybhE and AJ12739 P_{tac-10000} strains, respectively. These strains were each cultivated at 37° C. for 18 hours in a nutrient broth with 25 mg/l chloramphenicol, and 0.3 ml of the obtained culture was inoculated into 3 ml of a fermentation medium containing 25 mg/l chloramphenicol in a 20×200 mm test tube, and cultivated at 34° C. for 24 hours with a rotary shaker. After the cultivation, the amount of phenylalanine which had accumulated in the medium was determined by TLC. 10×15 cm TLC plates coated with 0.11 mm layers of Sorbfil silica gel without fluorescent indicator (Stock Company Sorbpolymer, Krasnodar, Russia) were used. Sorbfil plates were developed with a mobile phase: propan-2-ol:ethylacetate:25% aqueous ammonia:water=40:40:7:16 (v/v). A solution (2%) of ninhydrin in acetone was used as a visualizing reagent.

The composition of the fermentation medium (g/l):

| | |
|---|---|
| Glucose | 40.0 |
| (NH₄)₂SO₄ | 16.0 |
| K₂HPO₄ | 0.1 |
| MgSO₄.7H₂O | 1.0 |
| FeSO₄.7H₂O | 0.01 |
| MnSO₄.5H₂O | 0.01 |
| Thiamine HCl | 0.0002 |
| Yeast extract | 2.0 |
| Tyrosine | 0.125 |
| CaCO₃ | 20.0 |

Glucose and magnesium sulfate are sterilized separately. CaCO₃ dry-heat sterilized at 180° C. for 2 h. pH is adjusted to 7.0. Antibiotic is introduced into the medium after sterilization. The results are presented in Table 5.

TABLE 5

| E. coli strain | OD₆₀₀ | Amount of phenylalanine, g/l |
|---|---|---|
| AJ12739 | 18.2 ± 0.1 | 0.65 ± 0.4 |
| AJ12739 P_{tac-3900}-ybhE | 17.0 ± 0.3 | 0.9 ± 0.1 |
| AJ12739 P_{tac-10000}-ybhE | 16.3 ± 0.2 | 1.3 ± 0.1 |

It can be seen from Table 5 that the enhanced expression of pgl gene improved phenylalanine production of the AJ12739 strain.

### INDUSTRIAL APPLICABILITY

According to the present invention, production of L-amino acids such as L-tryptophan, L-phenylalanine, and L-tyrosine can be enhanced.

While the invention has been described in detail with reference to preferred embodiments thereof, it will be apparent to one skilled in the art that various changes can be made, and equivalents employed, without departing from the scope of the invention. All the cited references herein, including the foreign priority documents RU 2004105179 and RU2005101700, are incorporated as a part of this application by reference.

1. An L-amino acid-producing bacterium belonging to the Enterobacteriaceae family, wherein the bacterium has been modified to have enhanced activity of 6-phosphogluconolactonase.

2. The bacterium according to claim 1, wherein the expression of 6-phosphogluconolactonase gene has been enhanced by increasing a copy number of said gene, or by modifying an expression regulatory sequence of said gene.

3. The bacterium according to claim 1, wherein a native promoter of said gene is substituted with a more potent promoter.

4. The bacterium according to claim 1, wherein a native SD sequence of said gene is substituted with a more efficient SD sequence.

5. The bacterium according to claim 1, wherein the bacterium is selected from the genus selected from the group consisting of Escherichia, Enterobacter, Erwinia, Klebsiella, Pantoea, Providencia, Salmonella, Serratia, Shigella, and Morganella.

6. The bacterium according to claim 1, wherein the 6-phosphogluconolactonase gene is originated from Enterobacteriaceae family.

7. The bacterium according to claim 6, wherein the 6-phosphogluconolactonase gene encodes a protein selected from the group consisting of:
(A) a protein comprising the amino acid sequence shown in SEQ ID NO:2; and
(B) a protein comprising an amino acid sequence which includes deletion, substitution, insertion or addition of one or several amino acids in the amino acid sequence shown in SEQ ID NO:2, and which has an activity of 6-phosphogluconolactonase.

8. The bacterium according to claim 6, wherein said 6-phosphogluconolactonase gene is selected from the group consisting of:
(a) a DNA comprising a nucleotide sequence of the nucleotides 1 to 993 in SEQ ID NO:1; and
(b) a DNA which is hybridizable with a nucleotide sequence of the nucleotides 1 to 993 in SEQ ID NO: 1 or a probe which can be prepared from the nucleotide sequence under stringent conditions and encodes a protein having an activity of 6-phosphogluconolactonase.

9. The bacterium according to claim 8, wherein said stringent conditions comprise washing for 15 minutes at 60° C. at a salt concentration corresponding to 1×SSC and 0.1% SDS.

10. The bacterium according to claim 1, wherein said bacterium is further modified to have enhanced expression of a yddG open reading frame.

11. The bacterium according to claim 1, wherein said L-amino acid is an aromatic L-amino acid selected from the group consisting of L-tryptophan, L-phenylalanine, and L-tyrosine.

12. A method for producing an L-amino acid comprising cultivating the bacterium according to claim 1 in a culture medium, and collecting said L-amino acid from said culture medium.

13. The method according to claim 12, wherein said L-amino acid is selected from the group consisting of L-tryptophan, L-phenylalanine, and L-tyrosine.
